(19) United States
(12) Patent Application Publication
(10) Pub. No.: US 2015/0258020 A1
(43) Pub. Date: Sep. 17, 2015

(54) CHEWABLE TABLETS COMPRISING LEVONORGESTREL

(71) Applicants: Anekant Jain, Allentown, PA (US); Bruce Frank, Bala Cynwyd, PA (US); Nagesh Nanda, Guadalajara (ES); Orazio Luca Strusi, Leon (ES); Francisco Ferrer, Leon (ES); Celestino Ronchi, Milano (IT)

(72) Inventors: Anekant Jain, Allentown, PA (US); Bruce Frank, Bala Cynwyd, PA (US); Nagesh Nanda, Guadalajara (ES); Orazio Luca Strusi, Leon (ES); Francisco Ferrer, Leon (ES); Celestino Ronchi, Milano (IT)

(21) Appl. No.: 14/215,467

(22) Filed: Mar. 17, 2014

(51) Int. Cl. A61K 9/00; A61K 9/20; A61K 31/57

(52) U.S. Cl. CPC A61K 9/0056; A61K 31/57; A61K 9/2095; A61K 9/2077; A61K 9/2018; A61K 9/2054; A61K 9/2027; A61K 9/2013

(57) ABSTRACT

The present invention relates to a chewable tablet comprising levonorgestrel and a pharmaceutically acceptable excipient for use in contraception, a method of use of same and a kit containing same.

### BACKGROUND OF THE INVENTION

Levonorgestrel is a synthetic progestin used alone or in combination with an estrogen in various oral hormonal contraceptives, including emergency and post-coital contraceptives, and hormone replacement therapy (HRT).

Since the introduction of oral contraceptives (OCs), researchers have been directed toward developing new preparations that improve compliance with contraceptive regimens while maintaining efficacy.

U.S. Pat. No. 6,667,050 discloses a chewable, palatable oral contraceptive tablet comprising norethindrone, ethinyl estradiol and a chewable carrier. Use of the tablets in a method of female oral contraception is also disclosed.

Patent application WO 2007/146805 A discloses a chewable oral dosage form comprising ethinyl estradiol, norethindrone and a pharmaceutically acceptable excipient. The bioavailability of ethinyl estradiol is improved by enhancing its oral mucosal absorption thus avoiding the first pass metabolism.

Potential advantages of chewable tablets are mainly concerning patient acceptance and compliance. In general, chewable tablets are advantageous in that they combine the accuracy of dosage associated with tablets with the optimum bioavailability of suspensions.

Chewable dosage forms are designed to be mechanically disintegrated in the mouth to smaller pieces that can be subsequently swallowed. As the disintegration of chewable dosage forms is faster and complete compared to standard formulations that must disintegrate in the gastrointestinal tract, enhanced bioavailability of the active compound present in this type of dosage forms is usually expected.

However, in the development of chewable compositions a similar dissolution/disintegration profile to the standard oral non-chewable formulations is required because they might be swallowed by a patient without proper chewing. The dissolution test should be carried out with the intact tablet and not crushing or powdering it to simulate the worst-case scenario where the patient swallows the tablet without chewing it.

This is of special relevance in the case of oral contraceptives wherein if the entire dose of contraceptive is not released as a result of a slower disintegration of the dosage form, unintended pregnancy could result.

In general, immediate release contraceptives should be rapidly dissolved, meaning that they should release at least 60% of its content in 5 minutes and at least 80% of its content after 10 minutes.

The applicant has found that when levonorgestrel is formulated in a chewable tablet as disclosed in U.S. Pat. No. 6,667,050, it shows a much lower solubility profile compared with standard non-chewable pharmaceutical tablets and thus, it is not suitable for its administration as a contraceptive. The use of micronized levonorgestrel having a particle size D₉₀ ranging from 5 microns to 15 microns, commonly used in standard levonorgestrel non-chewable oral dosage formulations does not solve this problem.

Thus, there is still need of the development of a chewable tablet of levonorgestrel having the advantages of the chewable dosage forms, while assuring the efficacy of the formulation in case of administration without proper chewing.

The inventors have surprisingly found that an effective chewable contraceptive formulation comprising levonorgestrel as an active ingredient may be achieved by providing levonorgestrel with a particle size D₉₀ of less than 4 microns.

Additionally, it is expected that the chewable formulation according to the present invention also provides a maximum absorption of levonorgestrel, and ethinyl estradiol (if present) through the oral mucosa when the tablet is chewed.

The present invention provides a chewable dosage form comprising levonorgestrel and a pharmaceutically acceptable excipient, wherein levonorgestrel has a particle size D₉₀ of less than about 4 microns, preferably as measured by laser light diffraction and the process of its preparation.

### SUMMARY OF THE INVENTION

One aspect of the invention relates to a chewable tablet comprising levonorgestrel and at least one pharmaceutically acceptable excipient, wherein levonorgestrel has a particle size D₉₀ of less than about 4 microns as preferably measured by laser light diffraction. Other methods of measuring particle size are contemplated by the present invention and are known to those skilled in the art.

Also provided is a process for the preparation of chewable tablet of levonorgestrel, the process comprising:
a) blending levonorgestrel with one or more pharmaceutically acceptable excipients, and
   b) then forming the tablet by compression of the blend, wherein levonorgestrel has a particle size D₉₀ of less than about 4 microns as preferably measured by laser light diffraction.

Also provided is a method of female oral contraception, the method comprising the administration of a chewable tablet comprising a contraceptively acceptable amount of levonorgestrel, wherein levonorgestrel has a particle size D₉₀ of less than about 4 microns as preferably measured by laser light diffraction. The term female herein refers to any female species in need of contraception, e.g., animal, mammal, human, bird, etc.

### BRIEF DESCRIPTION OF THE DRAWINGS

FIG. 1 depicts the dissolution profile obtained for the chewable tablet of example 1.

FIG. 2 depicts the dissolution profile obtained for the chewable tablet of example 2.

### DESCRIPTION OF THE INVENTION

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

As used herein, “chewable dosage form” or “chewable tablet” means a dosage form or a tablet that is designed to be mechanically disintegrated in the mouth to smaller pieces that can be subsequently swallowed.

Several analytical tools are available to determine particle size distribution of a sample. Laser diffraction has been chosen as the preferable analytical method for measuring the particle size of levonorgestrel according to the invention. Other types of particle size measurement known to those skilled in the art include, but are not limited to microscopy, sieving, sedimentation techniques, optical and electrical sensing zone method and laser light scattering techniques.

As used herein, D10, D50 and D90 is the size, in microns, below which, respectively 10%, 50% and 90% of the particles by volume are found, i.e., a D90 of 4 microns means 90% of particles of the sample is below 4 microns in size, a D50 of 3 microns means that 50% of the particles of the sample is below 3 microns and D10 of 2 microns means that 10% of the particles of the sample is below 3. Evidently, D10, D50 and D90 do not include the zero value.

Levonorgestrel according to the present invention may be provided in particulate form, i.e., as a mixture of particles, or in spray dried form alone or as a mixture with a pharmaceutically acceptable excipient.

As used herein, the term “spray-dried levonorgestrel” or “spray-dried mixture of levonorgestrel with a pharmaceutically acceptable excipient”, unless otherwise specified, refers to levonorgestrel or a mixture of levonorgestrel with a pharmaceutically acceptable excipient that has been subjected to a spray-drying process.

The present invention provides a chewable dosage form of levonorgestrel, a process for its production and a method of oral contraception comprising the administration of a chewable dosage form comprising levonorgestrel.

One embodiment of the present invention is a chewable tablet comprising levonorgestrel and at least one pharmaceutically acceptable excipient, wherein levonorgestrel has a particle size D₉₀ of less than 4 microns as preferably measured by laser light diffraction.

Preferably, levonorgestrel according to the present invention has a particle size D₉₀ of less than 3 microns, more preferably of less than 2 microns as preferably measured by laser light diffraction.

A further embodiment of the invention is a chewable tablet comprising levonorgestrel and at least one pharmaceutically acceptable excipient, wherein levonorgestrel has a particle size D₉₀ of less than 4 microns and D₅₀ of less than about 2.5 microns as preferably measured by laser light diffraction.

Preferably, levonorgestrel according to the present invention has a particle size D₉₀ of less than about 3 microns and D₅₀ of less than about 2.0 microns, more preferably D₉₀ of less than about 2 microns and D₅₀ of less than 1 microns as preferably measured by laser light diffraction.

A further embodiment of the present invention is a chewable tablet comprising levonorgestrel and at least one pharmaceutically acceptable excipient, wherein levonorgestrel has a particle size D₉₀ of less than about 4 microns and D₅₀ of less than about 2.5 microns and D₁₀ of less than 1 micron as preferably measured by laser light diffraction.

Preferably, levonorgestrel according to the present invention has a particle size D₉₀ of less than about 3 microns, D₅₀ of less than about 2.0 microns and D₁₀ of less than 0.8 microns, more preferably D₉₀ of less than about 2 microns, D₅₀ of less than 1.5 microns and D₁₀ of less than about 0.5 microns as preferably measured by laser light diffraction.

Levonorgestrel having a particle size according to the present invention may be provided by any method known in the art, such as milling, micronization, spray drying, crystallization, crystallization via sonication, grinding or cryogrinding.

In a preferred embodiment levonorgestrel according to the present invention may be produced by spray-drying levonorgestrel with a suitable solvent.

In a further preferred embodiment of the invention levonorgestrel according to the invention is produced by spray-drying a mixture of levonorgestrel with a pharmaceutically acceptable excipient in a suitable

Thus, the present invention provides a chewable tablet of levonorgestrel and at least one pharmaceutically acceptable excipient, wherein levonorgestrel is spray-dried levonorgestrel.

The present invention also provides a chewable tablet of levonorgestrel and at least one pharmaceutically acceptable excipient, wherein levonorgestrel is provided as a spray-dried mixture of levonorgestrel with a pharmaceutically acceptable excipient.

The term “spray-dried levonorgestrel” or “spray-dried mixture of levonorgestrel with a pharmaceutically acceptable excipient” as used herein, unless otherwise specified, refers to levonorgestrel or a mixture of levonorgestrel with a pharmaceutically acceptable excipient that has been subjected to a spray-drying process.

The chewable tablet according to the invention can be used in an emergency or post-coital oral contraceptive, or in an oral contraceptive in conjunction with an contraceptive regimen. As used herein, an oral contraceptive regimen refers to any of a monophasic, biphasic, multiphasic, extended or flexible oral contraceptive regimens.

The dosage of levonorgestrel in the chewable tablet would be that conventionally used in the art for emergency, post-coital or oral contraception.

Levonorgestrel may be present in an amount from about 0.01% to 40%, preferably from about 0.05% to 30%, more preferably from 0.1% to 20% of the total weight of the composition, although these amounts can vary depending upon the conditions of use.

Typically, the amount used of levonorgestrel is between 0.01 to 2 mg, preferably 0.05 to 1 mg, particularly preferred 0.1 mg.

The chewable tablet of the invention may, if desired, include further active ingredients. In a preferred embodiment, said further active ingredient is an estrogen. Suitable estrogens are ethinylestradiol, mestranol, quinestranol, estradiol, estrone, estrane, estriol, estetrol and conjugated equine estrogens. Ethinylestradiol is particularly preferred.

When present, the estrogen may be present in an amount from about 0.01% to 1% of the total weight of the composition and these amounts may vary depending upon the circumstances of use.

Preferred amounts of ethinyl estradiol used according to the invention are for example 10 to 50 μg, preferably 10 to 30 μg, particularly preferred 20 μg.

In a preferred aspect of the invention, the chewable tablet of the invention comprises from 0.01 to 2 mg of levonorgestrel and 10 to 50 μg of ethinyl estradiol, preferably from 0.01 to 1 mg levonorgestrel and 10 to 30 μg ethinyl estradiol.

In a most preferred aspect of the invention, the chewable tablet of the invention comprises 0.1 mg levonorgestrel and 20 μg ethinyl estradiol.

When present, ethinyl estradiol preferably has a particle size D₉₀ of less than about 4 microns, D₅₀ of less than 3 microns and D₁₀ of less than about 2 microns.

The pharmaceutical excipient may be selected from diluents, binders, lubricants, disintegrants, flavoring agents, coloring agents, stabilizers, surfactants, glidants, plasticizers, preservatives, antiadherent and sweeteners.

In the manufacture of chewable tablets, it has to be ensured that that the tablets are easily crushed by chewing and that the tablets are palatable.

Ideally chewable formulations should have a smooth texture upon disintegration, pleasant taste and no bitter or unpleasant after taste.

Sweeteners, both naturally occurring and synthetic, are one type of functional excipients commonly used in chewable tablet formulations to mask unpleasant tastes.

Suitable sweeteners may be selected from aspartame, dextrose, sucralose, lactose, glucose, fructose, sucrose, xylitol, sorbitol, mannitol and cyclamate.

Sweeteneers may be present in an amount from about 10% to about 50%, preferably from about 12% to 40%, and more preferably from about 15 to 30% of the total weight of the composition.

Suitable flavouring agents include fruit and plant flavours, for example, orange, anise, mint, etc.

Flavouring agent may be present in an amount from about 2% to about 25%, preferably from about 5% to 20%, and more preferably from about 8 to 15% of the total weight of the composition.

Suitably, the dosage form according to the invention comprises one or more diluents. Suitable diluents include corn starch, microcrystalline cellulose, powdered cellulose, silicified cellulose, lactose monohydrate, anhydrous lactose, mannitol, sorbitol, xylitol sucrose, fructose, dextrose, calcium carbonate, calcium phosphate dibasic, calcium phosphate tribasic, calcium sulfate, maltose and maltodextrin and/or mixtures thereof.

Preferably lactose monohydrate and microcrystalline cellulose are used.

Diluents may be present in an amount from about 30% to about 95%, preferably from about 40% to 90%, and more preferably from about 50 to 87% of the total weight of the composition.

Suitably, the dosage form according to the invention comprises a binder. The binding agent is selected from the group consisting of hydroxypropyl cellulose, hydroxypropyl methyl cellulose, hydroxyethyl cellulose, methylcellulose, polyvinylpyrrolidone, sodium carboxymethyl cellulose, calcium carboxymethyl cellulose, maize starch, patato starch, pregelatinized starch, Polyethylene glycol (include all the grades), alginate, Chitosan and/or mixtures thereof. Preferably polyvinylpyrrolidone is used.

Binders may be present in an amount from about 0.1% to about 10% by weight, preferably from about 0.2% to about 9% by weight, and more preferably from about 0.5% to 7.5% by weight of the total weight of the composition.

Disintegrating agents may be selected from the group consisting of low-substituted hydroxypropyl cellulose, sodium carboxymethyl cellulose, calcium carboxymethyl cellulose, crospovidone, sodium croscarmellose, cellulose microcrystalline, maize starch, potato starch, pregelatinized starch, sodium starch glicolate and/or mixtures thereof. Preferably sodium croscarmellose is

Disintegrating agents may be present in an amount from about 1% to about 15% by weight, preferably from about 2% to about 12% by weight, and more preferably from about 3% to about 10% by weight of the total weight of the composition.

Lubricants may be selected from the group consisting of talc, alkaline earth salts of stearic acid, specially magnesium and calcium stearate, stearic acid, glycerin palmitostearate, stearyl fumarate, and/or mixtures thereof. Magnesium stearate is most preferred as lubricant.

The lubricant may be present in an amount from about 0% to about 5% by weight, preferably from about 0% to about 3% based on the total weight of the composition.

Suitable antiadherent agents include colloidal silicon dioxide and talc.

The antiadherent agent may be present in an amount from about 0% to about 5% by weight, preferably from about 0% to about 3% based on the total weight of the composition.

Suitable colouring agents, which may be incorporated into the tablets of the invention, may be selected from those approved for oral use.

Chewable tablets are usually uncoated, but if desired, the tablets may be coated using conventional methods known to a person skilled in the art, as those described in Remington: The Science and Practice of Pharmacy, 20th Edition, Philadelphia, Lippincott, Williams & Wilkins, 2000 [ISBN 0 683 306472].

Suitable surfactant agents may be selected from the group consisting of ionic surfactants, such as Sodium Lauryl Sulfate, Phospholipids, Glycerol Monooleate, Docusate sodium, or non-ionic surfactants, such as polisorbate 80, Polyoxyethylene Sorbitan Fatty Acid Esters, Polyoxyethylene Stearates, poloxamer, Polyoxyethylene Alkyl Ethers.

The surfactant agent may be present in an amount from about 0% to about 10% by weight, preferably from about 0% to about 5% based on the total weight of the composition.

According to a preferred embodiment the chewable tablet according to the invention comprises from about 0.05 mg to 0.15% levonorgestrel

According to yet another embodiment of the present invention a process for the preparation of chewable tablet comprising levonorgestrel, the process comprising:
a) providing levonorgestrel having a particle size D₉₀ of less than about 4 microns as preferably measured by laser light diffraction;
b) blending levonorgestrel of step a) with one or more pharmaceutically acceptable excipients; and
c) compression of the blend into tablets.

In a preferred embodiment levonorgestrel according to the present invention may be provided by spray-drying levonorgestrel with a suitable solvent.

Solvents suitable for spray-drying can be any organic solvent in which levonorgestrel is soluble. Preferred solvents include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, and 1,1,1-trichloroethane. Lower volatility solvents such as dimethyl acetamide or dimethylsulfoxide can also be used. Mixtures of solvents can also be used.

Spray-drying processes and spray-drying equipment are described generally in Perry's Chemical Engineers' Handbook, pages 20-54 to 20-57 (Sixth Edition 1984). More details on spray-drying processes and equipment are reviewed by Marshall, “Atomization and Spray-Drying,” 50 Chem. Eng. Prog. Monogr. Series 2 (1954), and Masters, Spray Drying Handbook (Fourth Edition 1985).

In a further preferred embodiment of the invention levonorgestrel according to the invention is provided by spray-drying a mixture of levonorgestrel with a pharmaceutically acceptable excipient in suitable solvent.

Typically, the ratio of levonorgestrel:excipient in the mixture may vary from about 1:1 to 1:10, preferably from about 1:2 to 1:8, by weight.

Preferably, the excipient is a polymer. Suitable polymers include polivinylpirrolidone, hydroxipropylmethyl cellulose (HPMC) and hydroxipropylmethyl acetate succinate. A preferred polymer is polivinylpirrolidone.

Another embodiment of the present invention is a process for the preparation of chewable tablet comprising levonorgestrel, the process comprising:
a) dissolving levonorgestrel in a suitable solvent;
b) spray-drying levonorgestrel from the solution obtained in step a;
c) blending spray-dried levonorgestrel of step b) with one or more pharmaceutically acceptable excipients; and
d) compression of the blend into tablets.

Another embodiment of the present invention is a process for the preparation of chewable tablet comprising levonorgestrel, the process comprising:
a) dissolving levonorgestrel and a pharmaceutically acceptable excipient in a suitable solvent;
b) spray-drying the solution obtained in step a);
c) optionally, blending the spray-dried mixture obtained in step b) with further pharmaceutical acceptable excipients; and
d) compression of the blend into tablets.

The hardness of the tablet may be any hardness that ensures that that the tablets are easily crushed by chewing. Additionally, the tablets must be hard enough to withstand mechanical stress during packaging, shipment, and handling by the consumer. The tablets of the invention preferably have a hardness of about 20N to about 40N, and preferably from 25N to 35N.

Also provided is an hormonal contraceptive method in human females, the method comprising the administration of a chewable tablet comprising a contraceptive acceptable amount of levonorgestrel, wherein levonorgestrel has a particle size D₉₀ of less than about 4 microns as preferably measured by laser light diffraction. This contraceptive method may also be utilized on other animals and mammals as needed.

Preferably, levonorgestrel according to the present invention has a particle size D₉₀ of less than about 3 microns, more preferably of less than about 2 microns as preferably measured by laser light diffraction.

Also provided is an hormonal contraceptive method in human females or other subjects in need of contraception, e.g., mammals, animals, etc., the method comprising the administration of a chewable tablet comprising a contraceptive acceptable amount of levonorgestrel, wherein levonorgestrel has a particle size D₉₀ of less than about microns as preferably measured by laser light diffraction and D₅₀ of less than about 2.5 microns as measured by laser light diffraction. This contraceptive method may also be utilized on other animals and mammals as needed.

Preferably, levonorgestrel according to the present invention has a particle size D₉₀ of less than about 3 microns and D₅₀ of less than about 2.0 microns, more preferably a D₉₀ of less than about 2 microns and a D₅₀ of less than about 1 microns as preferably measured by laser light diffraction.

Also provided is an hormonal contraceptive method in human females, the method comprising the administration of a chewable tablet comprising a contraceptive acceptable amount of levonorgestrel, wherein levonorgestrel has a particle size D₉₀ of less than about 4 microns as preferably measured by laser light diffraction, D₅₀ of less than about 2.5 microns and D₁₀ of less than about 1 micron as preferably measured by laser light diffraction. This contraceptive method may also be utilized on other animals and mammals as needed.

Preferably, levonorgestrel according to the present invention has a particle size D₉₀ of less than about 3 microns, D₅₀ of less than about 2.0 microns and D₁₀ of less than about 0.8 microns, more preferably D₉₀ of less than about 2 microns, D₅₀ of less than about 1.5 microns and D₁₀ of less than about 0.5 microns as preferably measured by laser light diffraction.

Typically, the chewable tablet of the invention is administered once daily as part of a contraceptive regimen. The regimen can comprise any number of days of administration of the tablets to the subject. Preferably, the regimen comprises administering the chewable tablets of the invention for about 21 to about 25 days, followed by about 3 to 7 days of placebo tablets not containing levonorgestrel or pill-free days.

Also provided is a kit for use in a hormonal contraceptive method in human females or other subjects in need of contraceptions such as mammals, animals, etc., said kit comprising at least 21 chewable tablets comprising from about 0.01 to 2 mg levonorgestrel wherein levonorgestrel has a particle size D₉₀ of less than about 4 microns as preferably measured by laser light diffraction. This contraceptive method may also be utilized on other animals and mammals as needed.

Also provided is a kit for use in a hormonal contraceptive method in human females or other subjects in need of contraceptions such as mammals, animals, etc., said kit comprising at least 21 chewable tablets comprising from about 0.01 to 1 mg levonorgestrel and from about 10 to 30 μg ethinyl estradiol wherein levonorgestrel has a particle size D₉₀ of less than 4 microns as preferably measured by laser light diffraction. This contraceptive method may also be utilized on other animals and mammals as needed.

The following examples are given to illustrate the invention in a sufficiently complete manner.

### EXAMPLES

### Example 1

5 1. Pre-Blend

Lactose monohydrate (3 mg) and ultra-micronized levonorgestrel are mixed and blended. The mixture is then passed through suitable mesh sieve.

In a separate container, lactose monohydrate (0.6 mg) and ethinylestradiol are mixed and blended. The mixture is then passed through suitable mesh sieve.

2. Blend

The two pre-bends of levonorgestrel and ethinyl estradiol obtained in the previous step are mixed and blended into a bin.

Then, remaining lactose, cellulose microcrystalline, povidone, croscarmellose sodium, mint flavor and xylitol are added to the mixture and blended. Magnesium stearate is then added to the mixture and blended.

3. Compression

The final blend is then compressed in 6.4 mm diameter punches to tablets.

| Tablet component | mg/Tablet | Formula % |
|---|---|---|
| Levonorgestrel | 0.1 | 0.1 |
| Povidone K30 | 0.5 | 0.5 |
| Ethinyl Estradiol | 0.02 | 0.02 |
| Mannitol | 22.8 | 22.8 |
| Lactose monohydrate | 36.5 | 36.5 |
| Microcrystalline cellulose | 20.0 | 20.0 |
| Croscarmellose sodium | 5.0 | 5.0 |
| Mint Flavour | 4.0 | 4.0 |
| Xylitol | 10.0 | 10.0 |
| Magnesium Stearate | 1.0 | 1.0 |
| Total weight | 100.0 | 100.0 |

Particle size determination of Levonorgestrel

1. Equipment
Diffractometer of laser light, Mastersizer 3000 or equivalent
Micro analytical balance or semimicro

2. Procedure

Weigh approximately 0.2-0.3g of the drug substance, and introduce to the dry accessory.

Instrumental Conditions:

Dispersion Unit: MAZ3500 and cell (AD01)

Refraction index: 1.5/1.5 (red light/blue light)

Absorption index: 1.0

Distribution Format:
Single mode
Enhanced sensitivity
Fine powder (activate)

Run time: 10 s

N° series: 1

Background run time: 10 s

Filter obscuration: 0.5-6%

Sampler settings:
Vibration feed rate: 50%
Dispersive air pressure: 2.5 bar

Feeder: 18 mm

| Batch | D10(μm) | D50(μm) | D90(μm) |
|---|---|---|---|
| Ultra-micronized | 0.2 | 1.0 | 1.1 |
| levonorgestel | | | |

### Example 2

Spray dried preparation of Levonorgestrel and an adjuvant:

| Material | Batch formula(g) | Batch formula(%) |
|---|---|---|
| Levonorgestrel | 10.000 | 16.67 |
| Polivinylpirrolidone | 50.00 | 83.33 |
| Ethanol 96% USP | 2000.00* | — |
| TOTAL | 2060.00 | 100 |

*eliminated during the process

Polivinylpirrolidone (PVP) is dissolved in 96% USP ethanol using a mechanical stirrer until a clear solution is reached. Once all the PVP is dissolved Levonorgestrel is added to the solution and stirred until no solid particles are detected in the solution.

The final solution contains 0.5% w/w of active and a ratio of 1:5 of Levonorgestrel:PVP.

The solution prepared is spray-dried using a spray dry Buchi set up with the parameters stated in the table below.

| Parameters | Target |
|---|---|
| Aspiration (%) | 100 |
| Inlet Temp. (° C.) | 115(Range 110-120) |
| Pump (%) | 60 |
| Nebulization P. (mm) | 60 |
| Product Temp. (° C.) | 60(Range 30-85) |

Particle Size of Levonorgestrel—PVP Spray Dried Particles

| Batch | D10(μm) | D50(μm) | D90(μm) |
|---|---|---|---|
| Spray dried levonorgestrel- | 0.24 | 1.57 | 3.75 |
| PVP mixture | | | |

The spray dried powder is used in the manufacturing of tablets of Levonorgestrel 0.1 mg/EE 0.02 mg having the following formula:

| Tablet component | | Mg/Tablet | Formula % |
|---|---|---|---|
| Spray-dried | Levonorgestrel | 0.1 | 0.60 |
| mixture | Povidone K30 | 0.5 | |

| | | |
|---|---|---|
| Ethinyl Estradiol | 0.02 | 0.02 |
| Mannitol | 22.8 | 22.8 |
| Lactose monohydrate | 36.5 | 36.5 |
| Microcrystalline cellulose | 20.0 | 20.0 |
| Croscarmellose sodium | 5.0 | 5.0 |
| Mint Flavour | 4.0 | 4.0 |
| Xylitol | 10.0 | 10.0 |
| Magnesium Stearate | 1.0 | 1.0 |
| Total weight | 100.0 | 100.0 |

Dissolution Method

For all examples, the chewable tablet was tested for dissolution of levonorgestrel at the following experimental conditions:

Apparatus: Apparatus II equipped with paddle

Speed: 75 rpm

Medium: tween 80 5ppm

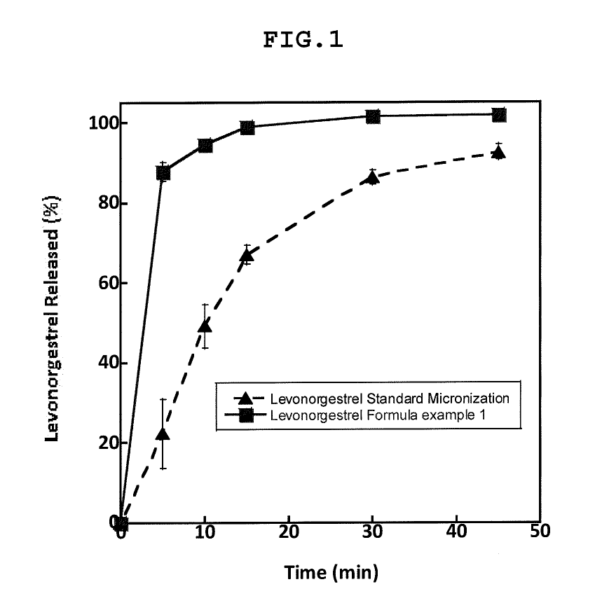

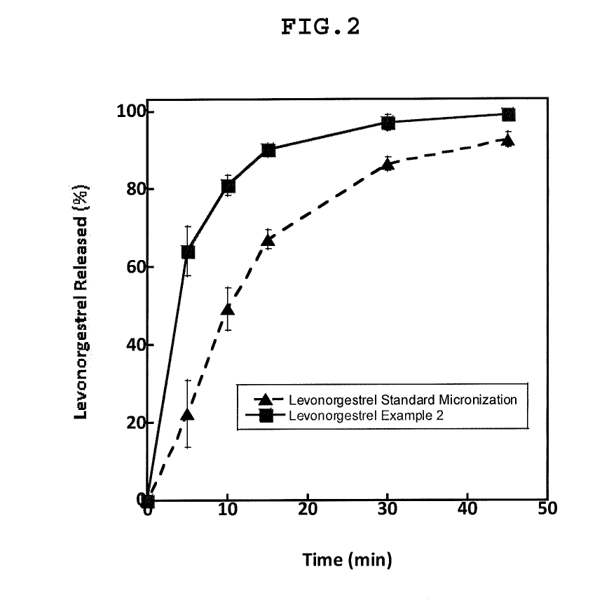

The dissolution profile obtained for the formulations described in examples 1 and 2 compared with the dissolution profile of the same formulation comprising levonorgestrel with the particle size disclosed in the table below are disclosed in FIGS. 1 and 2.

| Batch | D10(μm) | D50(μm) | D90(μm) |
|---|---|---|---|
| Levonorgestrel Standard | 1.29 | 6.02 | 12.1 |
| micronization | | | |

We claim:

1. A chewable tablet comprising levonorgestrel and at least one pharmaceutically acceptable excipient, wherein levonorgestrel has a particle size D₉₀ of less than about 4 microns.

2. A chewable tablet according to claim 1, wherein levonorgestrel has a particle size D₉₀ of less than about 3 microns, preferably less than about 2.

3. A chewable tablet according to claim 1, wherein levonorgestrel is spray-dried levonorgestrel.

4. A chewable tablet according to claim 1, wherein levonorgestrel is provided as a spray-dried mixture of levonorgestrel with a pharmaceutically acceptable excipient.

5. A chewable tablet according to claim 1, wherein the amount of levonorgestrel is between about 0.01 to 2 mg.

6. A chewable tablet according to claim 1, wherein the tablet further comprises from about 10 to 50 μg of ethinylestradiol.

7. A chewable tablet according to claim 6, wherein the tablet comprises about 0.1 mg levonorgestrel and about 20 μg ethinyl estradiol.

8. A process for the preparation of a chewable tablet comprising levonorgestrel, the process comprising:
a) providing levonorgestrel having a particle size D₉₀ of less than about 4 microns;
b) blending levonorgestrel of step a) with one or more pharmaceutically acceptable excipients to form a blend; and
c) compression of the blend into tablets.

9. A process according to claim 8, wherein levonorgestrel is provided by spray-drying levonorgestrel with a suitable solvent.

10. A process according to claim 9, wherein levonorgestrel is provided by spray-drying a mixture of levonorgestrel with a pharmaceutically acceptable excipient in suitable solvent.

11. A process for the preparation of a chewable tablet comprising levonorgestrel, the process comprising:
a) dissolving levonorgestrel in a suitable solvent to form a solution;
b) spray-drying levonorgestrel from the solution obtained in step a);
c) blending the spray-dried levonorgestrel of step b) with one or more pharmaceutically acceptable excipients to form a blend; and
d) compression of the blend into tablets.

12. A process for the preparation of a chewable tablet comprising levonorgestrel, the process comprising:
a) dissolving levonorgestrel and a pharmaceutically acceptable excipient in a suitable solvent to form a solution;
b) spray-drying the solution obtained in step a);
c) optionally, blending the spray-dried mixture obtained in step b) with further pharmaceutical excipients to form a blend; and
d) compression of the blend into tablets.

13. A process according to claim 12, wherein the amount of levonorgestrel:excipient in the mixture of step a) is from about 1:1 to 1:10, preferably from about 1:2 to 1:8, by weight.

14. An hormonal contraceptive method in a subject in need of contraception, the method comprising the administration of a chewable tablet comprising a contraceptive acceptable amount of levonorgestrel, wherein levonorgestrel has a particle size D₉₀ of less than about 4 microns as measured by laser light diffraction.

15. An hormonal contraceptive method according to claim 14, wherein the amount of levonorgestrel is between about 0.01 to 2 mg.

16. An hormonal contraceptive method according to claim 15, wherein the chewable tablet further comprises from about 10 to about 30 μg ethinyl estradiol.

17. A kit for use in a hormonal contraceptive method in a subject in need of contraception, said kit comprising at least 21 chewable tablets comprising from about 0.01 to 2 mg levonorgestrel wherein levonorgestrel has a particle size D₉₀ of less than about 4 microns.

18. A kit for use in a hormonal contraceptive method in a subject in need of contraception, said kit comprising at least 21 chewable tablets comprising from about 0.01 to 1 mg levonorgestrel and from about 10 to 30 μg ethinyl estradiol, wherein levonorgestrel has a particle size D₉₀ of less than about 4 microns.

19. The chewable tablet of claim 1 wherein the particle size of the levonorgestrel is measured by laser light diffraction.

20. The chewable tablet of claim 2 wherein the particle size of the levonorgestrel is measured by laser light diffraction.

21. The process of claim 8 wherein the particle size of the levonorgestrel is measured by laser light diffraction.

22. The kit of claim 17 wherein the particle size of the levonorgestrel is measured by laser light diffraction.

23. The kit of claim 17 wherein the particle size of the levonorgestrel is measured by laser light diffraction.

24. The kit of claim 14 wherein the subject in need of contraception is a human female.

25. The kit of claim 17 wherein the subject in need of contraception is a human female.

26. The chewable tablet of claim 1 wherein the particle size of the levonorgestrel is measured by microscopy, sieving, sedimentation techniques, optical and electrical sensing zone methods or laser light scattering techniques.

27. The chewable tablet of claim 2 wherein the particle size of the levonorgestrel is measured by microscopy, sieving, sedimentation techniques, optical and electrical sensing zone methods or laser light scattering techniques.

28. The process of claim 8 wherein the particle size of the levonorgestrel is measured by microscopy, sieving, sedimentation techniques, optical and electrical sensing zone methods or laser light scattering techniques.

29. The kit of claim 17 wherein the particle size of the levonorgestrel is measured by microscopy, sieving, sedimentation techniques, optical and electrical sensing zone methods or laser light scattering techniques.

30. The kit of claim 17 wherein the particle size of the levonorgestrel is measured by microscopy, sieving, sedimentation techniques, optical and electrical sensing zone methods or laser light scattering techniques.

31. The method of claim 14 wherein the subject in need of contraception is a female human.

32. The kit of claim 14 wherein the subject in need of contraception is a female human.

33. The kit of claim 14 wherein the subject in need of contraception is a female human.
